(19) United States
(12) Patent Application Publication
(10) Pub. No.: US 2022/0153788 A1
(43) Pub. Date: May 19, 2022

(54) METHOD AND PROCESS LINE FOR PRODUCING A DEWATERED GLUTEN-CONTAINING FRACTION

(71) Applicant: GEA MECHANICAL EQUIPMENT GMBH, Oelde (DE)

(72) Inventors: Andreas STORZER, Oelde (DE); Mario EMANUELE, Oelde (DE)

(21) Appl. No.: 17/603,627

(22) PCT Filed: Mar. 19, 2020
(86) PCT No.: PCT/EP2020/057558

§ 371 Date: Oct. 14, 2021

(30) Foreign Application Priority Data
Apr. 18, 2019 (DE) 10 2019 110 376.2

(51) Int. Cl. C07K 14/415; B04B 1/12; B04B 11/02; B04B 1/20; B01D 21/26; C07K 1/14

(52) U.S. Cl. CPC C07K 14/415; B04B 1/12; B04B 11/02; B04B 2001/2091; B01D 21/262; C07K 1/14; B04B 1/2016

(57) ABSTRACT

A method for producing a dewatered gluten-containing fraction involves providing a gluten-containing fraction, concentrating the gluten-containing fraction by centrifugal processing according to a determined dry substance content, adjusting the pH value and the temperature of the concentrated gluten-containing fraction by a temperature-control and metering unit, and dewatering the concentrated gluten-containing fraction.

### BACKGROUND AND SUMMARY OF THE INVENTION

Exemplary embodiments of the present invention relate to a method and process line for producing a dewatered gluten-containing fraction.

The starting material in the production of a dewatered gluten-containing fraction is preferably corn gluten (gluten) having a dry matter content of about 10 to 20 g/L separable solid.

U.S. Pat. No. 4,207,118 A describes a method for concentrating a gluten-containing fraction. However, dewatering with a decanter is not described here.

Other relevant prior art in the field is U.S. Pat. No. 7,452,425 B1 and EP 0 381 872 A1.

Based on the previously known prior art, exemplary embodiments of the present invention are directed to operating the method in the best product-optimized manner as possible and without major manual intervention.

A method according to the invention for producing a dewatered gluten-containing fraction comprises the following steps:

A Providing a gluten-containing fraction,

B Concentrating the gluten-containing fraction by centrifugal processing as a function of a determined dry matter content,

C Adjusting the pH value and temperature of the concentrated gluten-containing fraction by means of a temperature-control and metering unit;

D Dewatering the concentrated gluten-containing fraction.

The gluten-containing fraction provided in step A may preferably have 10 to 20 g/L of separable solid.

Concentration in step B takes place in the course of centrifugal processing. In this process, the fraction provided is separated into liquid and into a fraction with a higher concentration of solids. For concentration, a DM (dry matter) content is sensed, preferably online, i.e., in the process. This can be the final DM content or, particularly preferably, the DM content of the fraction directly at the outlet of a gluten concentrator.

To improve the flocculation and/or agglomeration properties, the pH value is adjusted and the temperature is adjusted. The pH value is preferably lowered and the temperature increased.

Finally, this fraction is dewatered. Dewatering can also be partial. Accordingly, the dewatered gluten-containing fraction has a residual moisture content even after dewatering.

To further specify a dewatered gluten-containing fraction, the dry matter content of this fraction after step D is at least 40 wt. %, preferably between 42-50 wt. %.

Dewatering of the concentrated gluten-containing fraction can be carried out as a function of a determined dry matter content. In this way, the dry matter content is also adjusted at this point so that the flowability of the fraction is maintained and clogging is prevented. At the same time, the highest possible degree of dewatering is to be achieved.

Concentration in step B may be performed as a function of a first determined dry matter content of the concentrated gluten-containing fraction after centrifugal processing in step B, and the dewatering of the concentrated gluten-containing fraction can take place as a function of a second determined dry matter content of the dewatered gluten-containing fraction after dewatering in step D. Thus, the dry matter content is always determined directly after the respective processing step and used to adjust the processing step.

The setting of the concentration condition is thus monitored. If the dry matter content after centrifugal processing exceeds or falls below a certain limit value, the processing for the subsequent product stream fed to the centrifugal processing is adjusted accordingly. The same applies to the dewatering of the concentrated gluten-containing fraction, which is performed as a function of a second determined dry matter content of a dewatered gluten-containing fraction, which is determined after the dewatering in step D. This adjustment does not enable the adjustment of the currently measured fraction, but it does enable the adjustment of another fraction subsequently fed to centrifugal processing.

Centrifugal processing in step B can preferably be carried out in a gluten concentrator, in particular in a nozzle separator.

Dewatering in step D can be carried out by a dewatering decanter, wherein the degree of dewatering is easily adjusted by setting the differential speed between a decanter screw and a decanter bowl of the dewatering decanter on the basis of the determined second dry matter content. Reducing the differential speed increases the dwell time of the product in the centrifugal field of the decanter, while increasing the differential speed has the opposite effect. Alternatively, or additionally, the bowl speed of the decanter can be changed. Increasing the bowl speed strengthens the centrifugal field, while reducing it has the opposite effect.

The gluten concentrator, in particular the nozzle separator, can have an outlet, wherein the outlet advantageously comprises a sensor for determining the dry matter content of the concentrated gluten-containing fraction in the outlet, and wherein the outlet comprises, in terms of flow, downstream of the sensor a regulating device, e.g., a valve, and a recirculation line into the gluten concentrator, wherein the regulating device recirculates the outflowing concentrated gluten-containing fraction or a partial flow thereof via the recirculation line until the measured value detected by the sensor corresponds to a setpoint value or exceeds it.

The dewatering decanter can also have a solids discharge, wherein the solids discharge comprises a sensor for determining the actual value of the dry matter content of the dewatered gluten-containing fraction in the solids discharge. If the actual value falls below a predetermined setpoint value, the differential speed between the decanter screw and the decanter bowl is adjusted until the measured value detected by the sensor corresponds to a setpoint value or exceeds it. Alternatively, the bowl speed can also be regulated accordingly.

To reduce pressure surges, a tank can be located upstream and downstream of the gluten concentrator.

The adjustment of the pH value in step C may be carried out to a range between 5.0 and 6.0, preferably between 5.3 and 5.8, wherein the adjustment is preferably carried out by adding an alkaline solution to the concentrated gluten-containing fraction, in particular a NaOH solution, as a function of a measured value of a pH sensor.

This makes it possible that a pH value between 5.3 and 5.8 can be set. A laboratory analysis of the dry matter content is not necessary due to the use of the inline pH sensor.

Since the effect of this change in dosage only becomes apparent with a time delay of several minutes in the solids discharge of the downstream dewatering decanter, the optimum setting of this controlled system by a single-manual process is very difficult, and fluctuations in the product properties in the gluten concentrator feed require constant adjustment of the amount of NaOH added. This is uncomplicated by using the pH sensor and a suitable machine or system controller.

The temperature can be set to a range between 55 to 63° C., preferably 58 to 60° C., wherein the setting is preferably effected by a regulated supply of hot water into a heat exchanger as a function of a measured value of a temperature sensor or a sensor for detecting a medium temperature. The concentrated gluten-containing fraction is passed through the aforementioned heat exchanger.

In this case, too, the use of the temperature sensor allows short-term changes in temperature to be compensated for by setting a heat exchanger accordingly.

The temperature can be adjusted by a setpoint comparison with a measured value of the temperature, which is detected by a sensor for determining the medium temperature. Similarly, the pH value can be set by adjusting the setpoint value with a measured value of the pH value, which is detected by a sensor for determining the pH value.

The adjustment of recirculation during concentration in step B, the adjustment of temperature and pH value in step C, and the adjustment of the differential speed between the decanter screw and the decanter bowl in step D can be performed in a user-friendly manner by a single control and evaluation unit.

Further according to the invention, a process line for producing a dewatered gluten-containing fraction comprises a gluten concentrator for concentrating the gluten-containing fraction in the course of centrifugal processing, a temperature-control and metering unit for adjusting the pH value and temperature of the concentrated gluten-containing fraction, and a dewatering decanter for dewatering the concentrated gluten-containing fraction. The gluten concentrator has an outlet with a sensor for detecting the dry matter content of the concentrated gluten-containing fraction in the outlet. Furthermore, in particular the outlet has a recirculation line for recirculating the concentrated gluten-containing fraction or a partial stream thereof as a function of the determined dry matter content. In this way, optimum and gentle concentration of the gluten or the gluten-containing fraction can be achieved. Corn gluten is particularly preferred for processing.

The gluten concentrator can preferably be designed as a nozzle separator with a disc stack. The nozzle separator also has a distributor below the disc stack. The recirculation line opens into the distributor below the disc stack.

The dewatering decanter has a solids discharge having a sensor for detecting the dry matter content of the dewatered gluten-containing fraction, wherein the process line comprises a control and evaluation unit, which is designed to set the differential speed between a decanter screw and a decanter bowl or the bowl speed of the dewatering decanter as a function of the determined dry matter content of the dewatered gluten-containing fraction.

The sensor for detecting the dry matter content of the concentrated gluten-containing fraction in the outlet and/or the sensor for detecting the dry matter content of the dewatered gluten-containing fraction can be designed as a measuring device for determining the viscosity of the respective fraction, in particular as a Coriolis flowmeter, as infrared sensors, in particular NIR sensors and/or as microwave-based sensors.

In particular, the aforementioned process line is used to carry out a method according to the invention.

### BRIEF DESCRIPTION OF THE DRAWING FIGURES

In the following, the invention is described in detail with reference to a specific exemplary embodiment and with reference to the accompanying figures. The individual features of the exemplary embodiment can also be adopted individually in other embodiment variants within the scope of the present invention. The drawings show by way of example:

FIG. 1 shows a process schematic of a method according to the invention for corn gluten dewatering; and

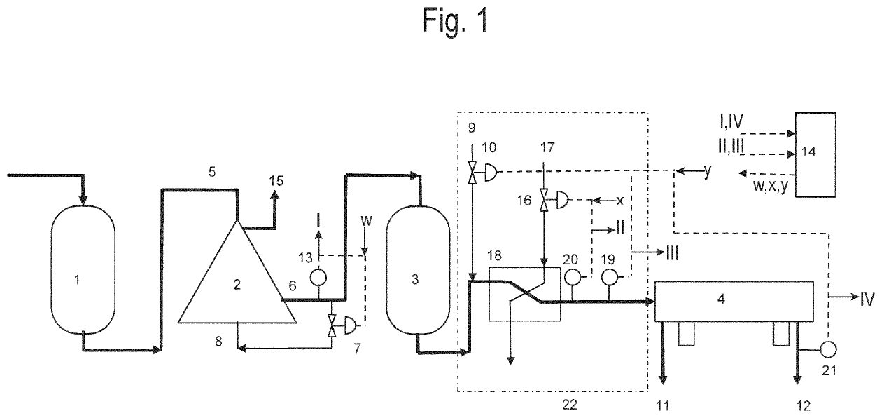

FIG. 2 shows a flowchart of a method according to the invention;

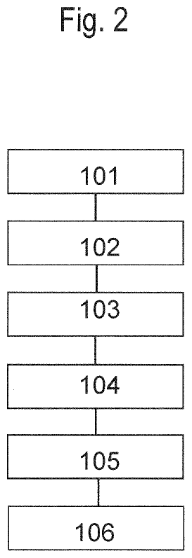

FIG. 3 shows a representation of a decanter suitable in the method according to the invention; and

FIG. 4 shows a representation of a separator suitable in the method according to the invention.

### DETAILED DESCRIPTION

Corn gluten dewatering according to the invention can be part of a starch extraction process.

In a first step 101, the provision of an aqueous corn gluten-containing fraction preferably having a dry matter content of about 10 to 20 g/L separable solid is carried out. The provision of the gluten-containing corn gluten dispersion can preferably be carried out as part of a starch recovery process, wherein starch is discharged as a product from the process and the aqueous gluten-containing gluten fraction is separately processed.

Gluten is a mixture of proteins, lipids, and carbohydrates after the removal of starch and soluble components. A large part thereof is gluten.

The provision of the aqueous corn gluten-containing fraction can take place in a tank 1.

In a second step 102, the corn gluten-containing fraction in the gluten is concentrated by a gluten concentrator 2, which is designed, for example, as a nozzle separator. For this purpose, the product is fed in via an inlet 5. The gluten concentrator has a first outlet 15 for the separated water phase and a second outlet 6 for a concentrated gluten-containing gluten fraction which emerges from the nozzles.

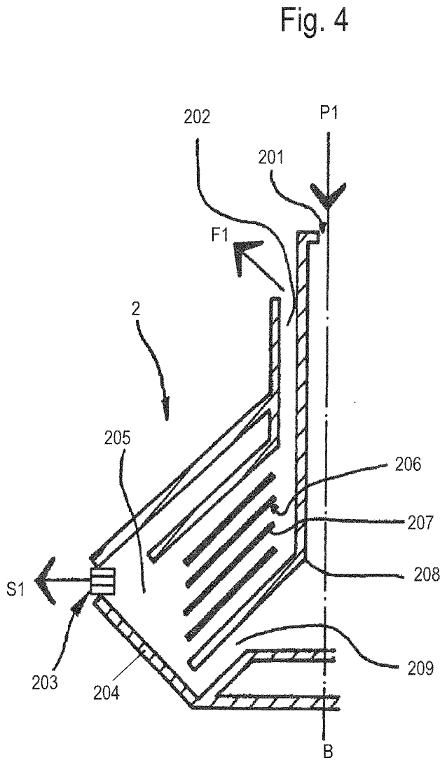

A suitable gluten concentrator 2 is shown as a centrifugal separator with a bowl 204 with an upright axis of rotation B in FIG. 4. It has a product inlet 201 for a starting material P1 and a product outlet 202 for a light phase F1, and discharge nozzles 203 for discharging a heavy phase S1 from the bowl 204. Inside the bowl 204, a disc stack 206 is arranged comprising a plurality of separating discs 207. The starting material P1 or the material to be separated is fed axially via an inlet pipe 208 into a distributor 209 and from there radially into a separating chamber 205 and into the spaces between the separating discs 207.

However, it is also possible to recirculate a portion of the concentrated corn gluten-containing fraction discharged at the discharge nozzles 203 into the gluten concentrator 2 through a recirculation line.

A regulating device 7 is arranged at this outlet 6, which returns a partial flow of the corn gluten-containing fraction concentrated from the nozzle separator 2 via a return line into the second inlet 8 and thus into the separator bowl. The quantity of the concentrated corn gluten-containing fraction returned in this way is adjusted by a regulating device 7. A corresponding regulating device 7 can, for example, be a regulating valve which is regulated as a function of the dry matter content measured by sensor 13.

The dry matter content (DM content) in the outlet 6 can thus be regulated by the nozzle separator 2 including the DM measurement 13 and the regulating valve 7. The detection can be carried out in different ways, for example by measuring the viscosity, e.g., by means of a Coriolis flowmeter, or by determination by means of one or more infrared sensors, in particular NIR sensors, microwave-based dry substance sensors, e.g., the MWTS series from hf sensor GmbH in Leipzig. The sensor 13 used is preferably a so-called inline sensor and the detection of the dry matter content can be an inline measurement.

Inline measurement, as distinguished from offline measurement, is a structure mounted along a production line with a sensor for continuous monitoring of the product or product flow passing the sensor.

The regulation of the DM content in % by weight in the gluten in the outlet 6 of the gluten concentrator 2, in particular of the nozzle separator, is carried out to a setpoint value in a range of preferably 35-50 g/L.

By measuring the dry matter content and comparing it with the aforementioned setpoint, it is possible to estimate whether and to what extent recirculation of a portion of the nozzle outlet takes place.

In a third step 103, the concentrated corn gluten-containing fraction is adjusted before dewatering. Here, the concentrated corn gluten-containing fraction is optionally passed through a buffer tank 3. The tank 1 upstream and the tank 3 downstream of the gluten concentrator 2 can each assume the function of a hydrophore and prevent pressure surges from the process to the gluten concentrator 2 and vice versa from the gluten concentrator 2 to the process.

A temperature-control and metering unit 22 is disposed downstream of the tank 3, which metering unit may include several devices and sensors. It preferably has a heat exchanger 18. The temperature-control and metering unit 22 can be used to regulate the temperature 105 of the gluten in the downstream decanter with the aid of a heat exchanger 18 in order to improve corn gluten dewatering. In this case, the product is raised from a temperature of about 45 to 50° C. in tank 3 to a temperature of 58 to 60° C.

The temperature-control and metering unit 22 may further comprise a metering device 10 for metering an alkaline solution, in particular an aqueous NaOH solution 9, into the corn gluten-containing concentrated fraction. The NaOH solution may be stored in a NaOH tank (not illustrated). The metering device 10 may be, for example, a control valve. The metering device 10 thus allows for the adjustment 104 of a pH value.

Preferably, the pH value of the corn gluten-containing fraction is adjusted, in particular regulated, in this step to a preferred pH value of 5.3 to 5.8. This can be carried out by adding NaOH solution to the feed of a subsequent dewatering decanter 4.

Furthermore, the temperature-control and metering unit 22 may comprise a regulating device 16, for example a valve for regulating the supply of hot water 17 into the heat exchanger 18. This can be used to regulate the temperature 105 of the gluten.

Furthermore, the temperature-control and metering unit 22 may comprise a sensor 20 for detecting the medium temperature of the concentrated gluten-containing fraction at the outlet of the heat exchanger 18, wherein the regulation of the feed quantity of hot water 17 is performed by the regulating device 16 on the basis of the measured values determined by the sensor 20.

Furthermore, the temperature-control and metering unit 22 comprises a pH sensor 19 for sensing the pH value of the concentrated corn gluten-containing fraction after pH adjustment 104.

The concentrated corn gluten-containing fraction with adjusted pH value and temperature is then fed to the dewatering decanter 4.

In a further step 106, the dewatering decanter 4 is used as a solid-bowl screw centrifuge for dewatering the gluten-containing fraction. The dewatering of the concentrated corn gluten-containing fraction is carried out in such a way that the discharged solid has a dry matter of preferably between 42 and 50% (in wt. %). This value can preferably be influenced by regulating the differential speed between the decanter bowl and the decanter screw. Additionally, or alternatively, the bowl speed and thus the g-value in the centrifugal field can also be varied. The dewatering decanter 4 has an outlet 11 for process water (light phase) and a solids discharge 12 for a dewatered or partially dewatered gluten-containing solids fraction (heavy phase).

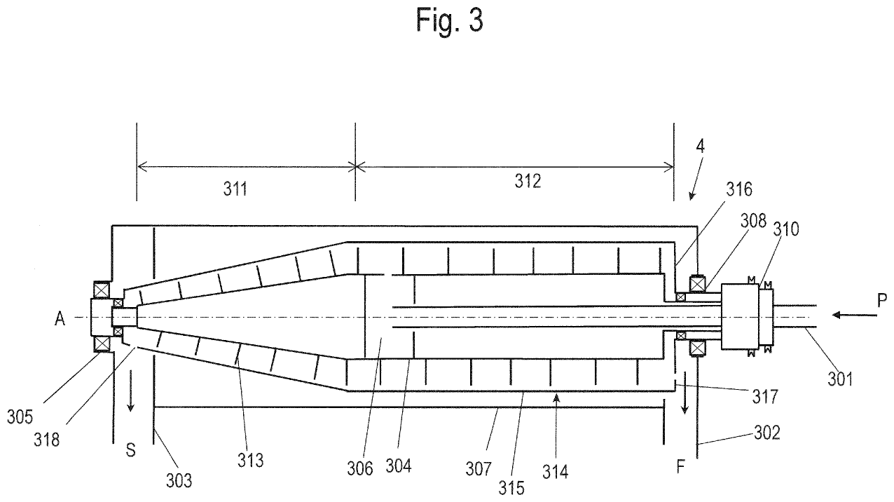

FIG. 3 shows a dewatering decanter 4 in the design of a solid-bowl screw centrifuge for use in the aforementioned method. The solid-bowl screw centrifuge has a frame and preferably housing 307 that is non-rotatable or non-rotating in operation, and a rotor 314, which is rotatable or rotating in operation. The rotor 314 comprises a rotatable bowl 315 with a horizontal axis of rotation D. For the rotatable bearing, corresponding bearings 308 and 305 are arranged at the end on the frame 307. However, the axis of rotation A can also be oriented differently, in particular vertically, in space. The rotor 314 also includes a screw 304 arranged in the bowl 315, the axis of rotation of which coincides with that of the bowl 315. In operation, the screw 304 can be rotated at a differential speed with respect to the bowl 315. A drive unit, which is not shown, controls and/or regulates the rotational speed of the bowl 315 and the screw 304 via one or more gears 310. The bowl 315 has a cylindrical section 312 and a conical section 311 axially adjoining the cylindrical section 312. The cylindrical section 312 is terminated by a substantially radially extending bowl cover 316. Here, the screw 304 also has a cylindrical section and a conical section axially adjoining the cylindrical section. It is disposed within the bowl 315. A feed pipe 301 extends into the bowl 315, here concentrically to the axis of rotation, and opens into a distributor 306, through which a suspension P to be processed can be fed radially into a centrifugal chamber 313 of the bowl 315. One or more liquid outlets 317 may be formed in or on the bowl cover 316, which open into a liquid discharge 302. This or these fluid outlets 317 may be formed in various ways, such as openings in the bowl cover 316 having a type of overflow weir, or in other ways, such as a peeling disc. At least one solids discharge opening 318 is formed at the end of the conical section 311, which opens into a solids discharge 303. In general, the bowl 315 is formed as a solid-wall bowl. At least one liquid phase F is then clarified from solids S in the rotating bowl 315.

The present method preferably makes use of four regulations, which are realized continuously and online and so that laboratory analysis is no longer necessary.

In a first regulation, the dry matter content in the nozzle outlet 6 of the gluten concentrator 2 is detected and measured online by a sensor 13. Depending on the measurement, part of the fraction on the nozzle side is returned to the feed of the bowl of the gluten concentrator until a setpoint value of a predetermined dry matter content at the sensor 13 is reached, which is preferably between 35-50 g/L.

Medium temperature of the concentrated corn gluten-containing fraction is adjusted to a setpoint of 58 to 60° C. in a second regulation.

The pH value of the concentrated corn gluten-containing fraction is adjusted to a target value of 5.3 to 5.8 in a third regulation.

Continuous online measurement of the dry matter content of the separated solids at the outlet of the dewatering decanter 4 is used to continuously adjust the differential speed between the decanter screw and the decanter bowl and/or the bowl speed with the aid of a control system containing corresponding controllers.

The measuring signals I, II, III, IV of the sensors 13, 19, 20 and 21 can each be processed by a separate control and evaluation unit and converted into control and/or regulating commands W, X, Y, Z with consideration to stored setpoint values in the aforementioned setpoint ranges. However, it is advantageous if the measurement signals are processed by a single control and evaluation unit 14 and converted accordingly. Corresponding signals and commands can be transmitted via signal lines or by radio.

The above-mentioned regulation allows the method to be operated particularly economically. Among other things, the consumption of NaOH can be reduced. The moisture content of the dewatered gluten at the solids discharge of the dewatering decanter is constantly low.

The overrun quality, i.e., the quality of the clear phase at outlet 15 of gluten concentrator 2, is more constant. Furthermore, this creates a much simpler and more stable mode of operation for both machines, which means that the operating personnel only have to intervene in the process to a small extent and incorrect process settings are avoided.

Although the invention has been illustrated and described in detail by way of preferred embodiments, the invention is not limited by the examples disclosed, and other variations can be derived from these by the person skilled in the art without leaving the scope of the invention. It is therefore clear that there is a plurality of possible variations. It is also clear that embodiments stated by way of example are only really examples that are not to be seen as limiting the scope, application possibilities or configuration of the invention in any way. In fact, the preceding description and the description of the figures enable the person skilled in the art to implement the exemplary embodiments in concrete manner, wherein, with the knowledge of the disclosed inventive concept, the person skilled in the art is able to undertake various changes, for example, with regard to the functioning or arrangement of individual elements stated in an exemplary embodiment without leaving the scope of the invention, which is defined by the claims and their legal equivalents, such as further explanations in the description.

### LIST OF REFERENCE SIGNS

1 Tank

2 Gluten concentrator

3 Tank

4 Dewatering decanter

5 Inlet

6 Outlet

7 Regulating device

8 Inlet

9 NaOH solution

10 Metering device

11 Outlet (process water)

12 Solids discharge

13 Sensor for detecting the DM content

14 Control and evaluation unit

15 Outlet

16 Regulating device

17 Hot water

18 Heat exchanger

19 pH sensor

20 Sensor for detecting the medium temperature

21 Sensor for detecting the DM content

22 Temperature-control and/or metering unit

101 Provision of an aqueous corn gluten-containing fraction

102 Concentration of the corn gluten-containing fraction

103 Adjustment of the concentrated corn gluten-containing fraction

104 pH value adjustment

105 Temperature regulation

106 Dewatering of the concentrated corn gluten-containing fraction.

I-IV Measurement signals

W-Z Control or regulation commands

201 Product inlet

202 Product outlet

203 Discharge nozzle

204 Bowl

205 Separating chamber

206 Disc stack

207 Discs

208 Inlet pipe

209 Distributor

B Axis of rotation

P1 Starting material

F1 Light phase

S1 Heavy phase

301 Inlet pipe

302 Liquid discharge

303 Solids discharge

304 Screw

305 Bearing

306 Distributor

307 Housing

308 Bearing

310 Gear

311 Conical section

312 Cylindrical section

314 Rotor

315 Bowl

316 Bowl cover

317 Liquid outlet

318 Solids discharge opening

P Suspension

S Solids

F Liquid phase

A Axis of rotation

1-18. (canceled)

19. A method for producing a dewatered gluten-containing fraction, the method comprising:
A. providing a gluten-containing fraction;
B concentrating the gluten-containing fraction by centrifugal processing as a function of a determined dry matter content to produce a concentrated gluten-containing fraction;
C adjusting a pH value and temperature of the concentrated gluten-containing fraction by a temperature-control and metering unit; and
D dewatering the pH value and temperature adjusted concentrated gluten-containing fraction.

20. The method of claim 19, wherein a dry matter content of the dewatered gluten-containing fraction is between 42-50 wt. %.

21. The method of claim 19, wherein the dewatering of the concentrated gluten-containing fraction is performed as a function of the determined dry matter content.

22. The method of claim 19, wherein the concentrating in step B is performed as a function of a first determined dry matter content of a concentrated gluten-containing fraction, which is determined after the centrifugal processing in step B, and the dewatering of the concentrated gluten-containing fraction is performed as a function of a second determined dry matter content of a dewatered gluten-containing fraction, which is determined after the dewatering in step D.

23. The method of claim 19, wherein the gluten-containing fraction in step A is provided as a corn gluten-containing fraction comprising 10 to 20 g/L of separable solid.

24. The method of claim 19, wherein the centrifugal processing occurs in a gluten concentrator, which is a nozzle separator.

25. The method of claim 22, wherein the dewatering in step D is performed by a dewatering decanter, wherein a degree of dewatering is performed by adjusting a differential speed between a decanter screw of the dewatering decanter and a decanter bowl or by adjusting a bowl speed of the dewatering decanter based on the determined second dry matter content.

26. The method of claim 19, wherein a gluten concentrator having an outlet is used as the gluten concentrator, which is a nozzle separator, wherein the outlet comprises a sensor that determines a dry matter content of the concentrated gluten-containing fraction in the outlet, and wherein the outlet comprises, downstream of the sensor in terms of flow, a regulating device and a recirculation line into an inlet of the gluten concentrator, wherein the regulating device recirculates part of the concentrated gluten-containing fraction via the recirculation line until a measured value detected by the sensor is equal to or exceeds a threshold.

27. The method of claim 25, wherein the dewatering decanter has a solids discharge, wherein the solids discharge has a sensor for determining the dry matter content of the dewatered gluten-containing fraction in the solids discharge, and wherein the differential speed between the decanter screw and the decanter bowl or the bowl speed is adjusted until a measured value detected by the sensor equals or exceeds a threshold.

28. The method of claim 24, wherein a first tank is arranged upstream of the gluten concentrator and a second tank is arranged downstream of the gluten concentrator, wherein the first or second tank includes the provided gluten-containing fraction or the dewatered fraction.

29. The method of claim 19, wherein the adjustment of the pH value in step C is performed to adjust the pH value to a range between 5.3 and 5.8, wherein the adjustment is performed by metering an NaOH solution into the concentrated gluten-containing fraction as a function of a measured value of a pH sensor.

30. The method of claim 19, wherein the adjustment the temperature is performed to adjust the temperature to a range between 58 to 60° C., wherein the adjustment the temperature is performed by a regulated supply of hot water into a heat exchanger as a function of a measured value of a sensor for detecting a medium temperature.

31. The method of claim 30, wherein the adjustment the temperature is performed by comparing a threshold with a measured value of the temperature, which is detected by the sensor for determining the medium temperature, and the adjustment of the pH value is performed by a setpoint adjustment with a measured value of the pH value, which is detected by a sensor for determining the pH value.

32. The method of claim 26, wherein the setting of the recirculation during concentration in step B, the setting of the temperature and the pH value in step C, and a differential speed between a decanter screw and a decanter bowl or a setting of a bowl speed in step D are performed by a single control and evaluation unit.

33. A production line for producing a dewatered gluten-containing fraction, the production line comprising:
a gluten concentrator configured to produce a concentrated gluten-containing fraction using centrifugal processing;
a temperature-control and metering unit configured to adjust a pH value and temperature of the concentrated gluten-containing fraction; and
a dewatering decanter configured to dewater the pH value and temperature adjusted concentrated gluten-containing fraction to produce a dewatered gluten-containing fraction,
wherein the gluten concentrator comprises an outlet having a sensor configured to determine a dry matter content of the concentrated gluten-containing fraction in the outlet and a recirculation line configured to recirculate the concentrated gluten-containing fraction into the inlet of the gluten concentrator as a function of the determined dry matter content.

34. The production line of claim 33, wherein the dewatering decanter has a solids discharge having a sensor configured to detect a dry matter content of the dewatered gluten-containing fraction, wherein the production line further comprises a control and evaluation unit configured to set a differential speed between a decanter screw and a decanter bowl or a bowl speed of the dewatering decanter as a function of the determined dry matter content of the dewatered gluten-containing fraction.

35. The production line of claim 34, wherein the sensor configured to detect the dry matter content of the concentrated gluten-containing fraction in the outlet or the sensor configured to detect the dry matter content of the dewatered gluten-containing fraction is a Coriolis flowmeter, a near-infrared sensor, or microwave-based sensor.
